(19) United States
(12) Patent Application Publication
(10) Pub. No.: US 2009/0274776 A1
(43) Pub. Date: Nov. 5, 2009

(54) Method for the Diagnosis and Treatment of Conditions Involving Aberrant Erythrocyte Potassium Levels

(75) Inventors: Maria Carolina Delgado, Doral, FL (US); Antonio Delgado Almeida, Valencia (VE)

Correspondence Address: Casimir Jones, S.C., 440 Science Drive, Suite 203, Madison, WI, 53711, US

(73) Assignee: THE REGENTS OF THE UNIVERSITY OF MICHIGAN, Ann Arbor, MI (US)

(21) Appl. No.: 12/297,840

(22) PCT Filed: Apr. 30, 2007
(86) PCT No.: PCT/US2007/010455

§ 371 Date: Apr. 13, 2009

Related U.S. Application Data
(60) Provisional application No. 60/795,880, filed on Apr. 28, 2006.

(51) Int. Cl. A61K 33/06; C12Q 1/02; A61P 9/12; C12M 1/34; A61K 33/00

(52) U.S. Cl. 424/722; 435/29; 435/287.1

(57) ABSTRACT

The present invention provides a systematic approach that allows health care providers (e.g., physicians) to use erythrocyte potassium measurements as an indicator of hypertension or risk of developing hypertension. In particular, the present invention provides systems and methods for treating conditions involving aberrant erythrocyte potassium levels (e.g., hypertension), preventing the onset of conditions involving aberrant erythrocyte potassium levels, identifying individuals at risk for developing hypertension, and evaluating the effectiveness of treatments for conditions involving aberrant erythrocyte potassium levels (e.g., hypertension).

The present application claims priority to U.S. Provisional Patent Application Ser. No. 60/795,880, filed Apr. 28, 2006, which is herein incorporated by reference in its entirety.

### FIELD OF THE INVENTION

The present invention provides a systematic approach that allows health care providers (e.g., physicians) to use erythrocyte potassium measurements as an indicator of hypertension or risk of developing hypertension and related conditions. In particular, the present invention provides systems and methods for treating conditions involving aberrant erythrocyte potassium levels (e.g., hypertension), preventing the onset of conditions involving aberrant erythrocyte potassium levels, identifying individuals at risk for developing hypertension, and evaluating the effectiveness of treatments for conditions involving aberrant erythrocyte potassium levels (e.g., hypertension).

### BACKGROUND

According to the Seventh Report of the Joint National Committee on the Prevention, Detection, Evaluation, and Treatment of High Blood Pressure, hypertension affects approximately 50 million individuals in the United States and approximately 1 billion worldwide. As the population ages, the prevalence of hypertension will increase even further unless broad and effective preventive measures are implemented. Recent data from the Framingham Heart Study suggest that individuals who are normotensive at age 55 have a 90 percent lifetime risk for developing hypertension.

The “Prevent and Control America's High Blood Pressure: Mission Possible” from the National Heart, Lung and Blood Institute, shows that high blood pressure can lead to heart disease, stroke, and kidney failure, the first, third and ninth causes of death in the United States. High blood pressure is a factor in 67 percent of heart attacks and 77 percent of strokes in the United States. High blood pressure precedes 74 percent of cases of heart failure in the United States, is the second leading cause of chronic kidney failure in the and creates a higher risk for mental deterioration and Alzheimer's.

High blood pressure causes more visits to doctors than any other condition. High blood pressure and its complications cost the U.S. economy more than $100 billion each year. Therefore, a great need exists for methods to successfully stratify, evaluate and treat pre-hypertensive and hypertensive patients in order to reduce both the enormous cost associated with hypertension, and consequential costs of cardiovascular diseases.

### SUMMARY OF THE INVENTION

The present invention provides a systematic approach that allows health care providers (e.g., physicians) to use erythrocyte potassium measurements as an indicator of hypertension or risk of developing hypertension and related conditions. In particular, the present invention provides systems and methods for treating conditions involving aberrant erythrocyte potassium levels (e.g., hypertension), preventing the onset of conditions involving aberrant erythrocyte potassium levels, identifying individuals at risk for developing hypertension, and evaluating the effectiveness of treatments for conditions involving aberrant erythrocyte potassium levels (e.g., hypertension).

In experiments conducted during the course of the present invention, a relationship between hypertension risk and erythrocyte potassium levels was identified. In particular, it was shown that individuals having erythrocyte potassium levels above a certain threshold (hereinafter, “erythrocyte potassium level threshold”) are at low risk for hypertension, while individuals having erythrocyte potassium levels below the erythrocyte potassium level threshold are at high risk for hypertension. The erythrocyte potassium level threshold is not limited to a particular value or range of values. For adult human individuals, the identified erythrocyte potassium level threshold was approximately 90 mmol/L per cell. For child human individuals, the identified erythrocyte potassium level threshold was approximately 94 mmol/L per cell.

The identified erythrocyte potassium level threshold values are not limited to a particular value or range of values. In some embodiments, “approximately 90 mmol/L per cell” describes erythrocyte potassium levels between 89 mmol/L per cell and 91 mmol/L per cell. In some embodiments, “approximately 94 mmol/L per cell” describes erythrocyte potassium levels between 93 mmol/L per cell and 95 mmol/L per cell.

In some embodiments, the erythrocyte potassium level threshold includes erythrocyte potassium level values higher or lower than “approximately 90 mmol/L per cell” (e.g., . . . 80 mmol/L per cell, 86 mmol/L per cell, 88 mmol/L per cell, 92 mmol/L per cell, 94 mmol/L per cell, 100 mmol/L per cell . . . ) and “approximately 94 mmol/L per cell” (e.g., . . . 84 mmol/L per cell, 90 mmol/L per cell, 92 mmol/L per cell, 96 mmol/L per cell, 98 mmol/L per cell, 104 mmol/L per cell . . . ). In some embodiments, the erythrocyte potassium level threshold varies depending on other criteria (e.g., an individual's related medical condition, an individual's genetic profile, etc.).

The present invention provides systems, methods, kits and devices that utilize the identified erythrocyte potassium level threshold in, for example, the monitoring, treatment, prevention, evaluation, and diagnosis of hypertension and risk of hypertension and related conditions.

In certain embodiments, the present invention provides a method of evaluating the effectiveness of a hypertension treatment for an individual, comprising a) administering the hypertension treatment to the individual, b) obtaining erythrocyte potassium levels for the individual, and c) evaluating the effectiveness of the hypertension treatment based upon the obtained erythrocyte potassium levels. In some embodiments, the method further comprises the step of obtaining a baseline erythrocyte potassium level for the individual prior to the administering of the hypertension treatment. In some embodiments, the obtaining erythrocyte potassium levels occurs during the course of the hypertension treatment. In some embodiments, the method further comprises the step of obtaining a post-treatment erythrocyte potassium level. In some embodiments, the method further comprises the step of adjusting or monitoring the treatment so as to maintain the erythrocyte potassium level at or above a desired erythrocyte potassium level.

In some embodiments, the individual is a human being over 20 years of age. In some embodiments, the desired erythrocyte potassium level is approximately 90 mmol/L per cell. In some embodiments, approximately 90 mmol/L per cell is 90 mmol/L per cell.

In some embodiments, the individual is a human being under 21 years of age. In some embodiments, the desired erythrocyte potassium level is approximately 94 mmol/L per cell. In some embodiments, the approximately 94 mmol/L per cell is 94 mmol/L per cell.

In some embodiments, an increase in the individual's erythrocyte potassium levels above approximately 90 mmol/L per cell during the hypertension treatment administration indicates the hypertension treatment is effective. In some embodiments, a decrease in the individual's erythrocyte potassium levels below approximately 90 mmol/L per cell during the hypertension treatment administration indicates the hypertension treatment is ineffective. In some embodiments, an increase in the individual's erythrocyte potassium levels above approximately 94 mmol/L per cell during the hypertension treatment administration indicates the hypertension treatment is effective. In some embodiments, a decrease in the individual's erythrocyte potassium levels below approximately 94 mmol/L per cell during the hypertension treatment administration indicates the hypertension treatment is ineffective.

In some embodiments, the obtaining erythrocyte potassium levels comprises a collection of a blood sample from the individual and analysis of the blood sample. In some embodiments, the analysis of the blood sample comprises measurement of the erythrocyte potassium levels. In some embodiments, the measurement is accomplished with a hand-held device.

In some embodiments, the hypertension treatment comprises life-style modification. In some embodiments, the life-style modification comprises one or more life-style modifications selected from the group consisting of a dietary change, a reduction in alcohol intake, an increase in aerobic activity, a reduction or elimination of nicotine intake, an adequate intake of dietary calcium and magnesium, and a reduction of sodium intake.

In some embodiments, the hypertension treatment comprises a pharmacological treatment. In some embodiments, the pharmacological treatment comprises one or more pharmacological treatments selected from the group consisting of administration of one or more potassium sparing drugs, administration of one or more aldosterone blocking drugs, administration of one or more potassium oral supplements, and administration of one or more anti-hypertensive drugs.

In some embodiments, the hypertension treatment comprises an experimental treatment.

In certain embodiments, the present invention provides a method of diagnosing risk for hypertension, comprising a) obtaining an adult individual's erythrocyte potassium level, and b) diagnosing the individual as being i) at low risk for hypertension if the erythrocyte potassium level is above approximately 90 mmol/L per cell, or ii) at high risk for hypertension if the erythrocyte potassium level is at or below approximately 90 mmol/L per cell. In some embodiments, the approximately 90 mmol/L per cell is 90 mmol/L per cell. In some embodiments, the method further comprises the step of administering a hypertension treatment to the individual if the adult individual is diagnosed as being at high risk for hypertension. In some embodiments, the adult individual is over 20 years of age.

In some embodiments, the hypertension treatment comprises life-style modification. In some embodiments, the life-style modification comprises one or more life-style modifications selected from the group consisting of a dietary change, a reduction in alcohol intake, an increase in aerobic activity, a reduction or elimination of nicotine intake, an adequate intake of dietary calcium and magnesium, and a reduction of sodium intake.

In some embodiments, the hypertension treatment comprises a pharmacological treatment. In some embodiments, the pharmacological treatment comprises one or more pharmacological treatments selected from the group consisting of administration of one or more potassium sparing drugs, administration of one or more aldosterone blocking drugs, administration of one or more potassium oral supplements, and administration of one or more anti-hypertensive drugs.

In some embodiments, the hypertension treatment comprises an experimental treatment.

In certain embodiments, the present invention provides a method of diagnosing risk for hypertension, comprising a) obtaining a child individual's erythrocyte potassium level, and b) diagnosing the individual as being i) at low risk for hypertension if the erythrocyte potassium level is above approximately 94 mmol/L per cell, or ii) at high risk for hypertension if the erythrocyte potassium level is at or below approximately 94 mmol/L per cell. In some embodiments, the approximately 94 mmol/L per cell is 94 mmol/L per cell. In some embodiments, the method further comprises the step of administering a hypertension treatment to the individual if the child individual is diagnosed as being at high risk for hypertension. In some embodiments, the child individual is under 21 years of age.

In some embodiments, the hypertension treatment comprises life-style modification. In some embodiments, the life-style modification comprises one or more life-style modifications selected from the group consisting of a dietary change, a reduction in alcohol intake, an increase in aerobic activity, a reduction or elimination of nicotine intake, an adequate intake of dietary calcium and magnesium, and a reduction of sodium intake.

In some embodiments, the hypertension treatment comprises a pharmacological treatment. In some embodiments, the pharmacological treatment comprises one or more pharmacological treatments selected from the group consisting of administration of one or more potassium sparing drugs, administration of one or more aldosterone blocking drugs, administration of one or more potassium oral supplements, and administration of one or more anti-hypertensive drugs.

In some embodiments, the hypertension treatment comprises an experimental treatment.

In certain embodiments, the present invention provides method of treating hypertension, comprising administering to an individual diagnosed as having hypertension a treatment configured to increase the individual's erythrocyte potassium level above a predetermined erythrocyte potassium level threshold, obtaining at least one measurement of the individual's erythrocyte potassium levels during the course of the treatment, and monitoring the effectiveness of the treatment through comparison of the measured erythrocyte potassium levels with the predetermined erythrocyte potassium level threshold. In some embodiments, the individual is a human being over 20 years of age. In some embodiments, the predetermined erythrocyte potassium level threshold is approximately 90 mmol/L per cell. In some embodiments, the approximately 90 mmol/L per cell is 90 mmol/L per cell. In some embodiments, the individual is a human being under 21 years of age. In some embodiments, the predetermined erythrocyte potassium level threshold is approximately 94 mmol/L per cell. In some embodiments, the approximately 94 mmol/L per cell is 94 mmol/L per cell.

In some embodiments, the treatment comprises life-style modification. In some embodiments, the life-style modification comprises one or more life-style modifications selected from the group consisting of a dietary change, a reduction in alcohol intake, an increase in aerobic activity, a reduction or elimination of nicotine intake, an adequate intake of dietary calcium and magnesium, and a reduction of sodium intake.

In some embodiments, the treatment comprises a pharmacological treatment. In some embodiments, the pharmacological treatment comprises one or more pharmacological treatments selected from the group consisting of administration of one or more potassium sparing drugs, administration of one or more aldosterone blocking drugs, administration of one or more potassium oral supplements, and administration of one or more anti-hypertensive drugs.

In some embodiments, the treatment comprises an experimental treatment.

In certain embodiments, the present invention provides a method of preventing the onset of hypertension, comprising administering to an individual at risk for developing hypertension a treatment configured to increase the individual's erythrocyte potassium level above a predetermined erythrocyte potassium level threshold, obtaining at least one measurement of the individual's erythrocyte potassium levels during the course of the treatment, and monitoring the effectiveness of the treatment through comparison of the measured erythrocyte potassium levels with the predetermined erythrocyte potassium level threshold.

In some embodiments, the individual is a human being over 20 years of age. In some embodiments, the predetermined erythrocyte potassium level threshold is approximately 90 mmol/L per cell. In some embodiments, the approximately 90 mmol/L per cell is 90 mmol/L per cell.

In some embodiments, the individual is a human being under 21 years of age. In some embodiments, the predetermined erythrocyte potassium level threshold is approximately 94 mmol/L per cell. In some embodiments, the approximately 94 mmol/L per cell is 94 mmol/L per cell.

In some embodiments, the treatment comprises life-style modification. In some embodiments, the life-style modification comprises one or more life-style modifications selected from the group consisting of a dietary change, a reduction in alcohol intake, an increase in aerobic activity, a reduction or elimination of nicotine intake, an adequate intake of dietary calcium and magnesium, and a reduction of sodium intake.

In some embodiments, the treatment comprises a pharmacological treatment. In some embodiments, the pharmacological treatment comprises one or more pharmacological treatments selected from the group consisting of administration of one or more potassium sparing drugs, administration of one or more aldosterone blocking drugs, administration of one or more potassium oral supplements, and administration of one or more anti-hypertensive drugs.

In some embodiments, the treatment comprises an experimental treatment.

In certain embodiments, the present invention provides a device configured to measure and/or monitor an individual's erythrocyte potassium levels. The present invention is not limited to a particular type or kind of device. In some embodiments, the device is a hand-held device. In some embodiments, the device is a desktop computer.

The devices of the present invention are not limited to a particular manner of measuring an individual's erythrocyte potassium levels. In some embodiments, the devices are designed to measure such differences with imaging agents (e.g., bioluminescence, fluorescence, etc.). In some embodiments, the devices are configured to utilize flame emission spectroscopy for purposes of measuring the individual's erythrocyte potassium level. In some embodiments, the device is configured to utilize potassium selective electrodes for purposes of measuring the individual's erythrocyte potassium level.

The devices of the present invention are not limited to a particular manner of reporting results derived with the device (e.g., the measured erythrocyte potassium level for the individual) (e.g., the monitoring of an individual's risk for hypertension). In some embodiments, the devices report derived results with a digital display. In some embodiments, the reporting is wireless communication to the Internet. In some embodiments, the reporting is wireless communication to a server. In some embodiments, the reporting is a textually based message displayed on the device. In some embodiments, the reporting is an audible message presented with the device.

In some embodiments, the devices have therein a processor for the calculating an individual's erythrocyte potassium level. In some embodiments, the processor is configured to interact (e.g., wireless) with software configured to accomplish the measuring of an individual's erythrocyte potassium level. In some embodiments, device has therein a processor for monitoring (e.g., comparing different erythrocyte potassium levels measured at different times). In some embodiments, the processor is configured to interact with software configured to accomplish the comparing of the measured erythrocyte potassium level with the predetermined erythrocyte potassium level threshold. In some embodiments, the devices are configured to interact (e.g., wireless) with a database containing information for a patient (e.g., a hospital database). In some embodiments, the devices have a memory for storing measured erythrocyte potassium levels over a period of time. In some embodiments, the memory is at least 100 Mb (e.g., 150 Mb, 700 Mb, 1 gig, 100 gigs, 1 terabyte, 100 terabytes). In some embodiments, the interacting is wireless communication to the Internet.

In certain embodiments, the present invention provides methods for monitoring an individual's risk for hypertension. The present invention is not limited to particular methods for monitoring an individual's risk for hypertension. In some embodiments, a device is used to monitor an individual's risk for hypertension, wherein the device is configured to a) receive a sample comprising erythrocytes from an individual; b) measure the erythrocyte potassium level within the erythrocytes; c) compare the measured erythrocyte potassium level with a predetermined erythrocyte potassium level threshold, wherein the measured erythrocyte potassium levels above the predetermined erythrocyte potassium level threshold represent a low risk for hypertension, wherein the measured erythrocyte potassium levels below the predetermined erythrocyte potassium level threshold represent a high risk for hypertension; and d) report the risk for hypertension.

In some embodiments, the individual is a human being over 20 years of age. In some embodiments, the predetermined erythrocyte potassium level threshold is approximately 90 mmol/L per cell. In some embodiments, the approximately 90 mmol/L per cell is 90 mmol/L per cell.

In some embodiments, the individual is a human being under 21 years of age. In some embodiments, the predetermined erythrocyte potassium level threshold is approximately 94 mmol/L per cell. In some embodiments, the approximately 94 mmol/L per cell is 94 mmol/L per cell.

In certain embodiments, the present invention provides kits for measuring an individual's erythrocyte potassium level from a blood sample. In some embodiments, the kit comprises a blood collection vessel and a device for measuring an individual's erythrocyte potassium level from a blood sample. In some embodiments, the device is a hand-held device. In some embodiments, the device is a desktop device. In some embodiments, the blood collection vessel is provided with a lancet with a cellulose strip having thereon antibodies for red blood cells.

### BRIEF DESCRIPTION OF THE DRAWINGS

FIG. 1 shows erythrocyte potassium levels in hypertensives, offspring and controls.

### DEFINITIONS

To facilitate an understanding of the present invention, a number of terms and phrases are defined below.

As used herein, the term “subject” or “individual” refers to any and all kinds or type of organisms. Such organisms preferably include, but are not limited to, mammals (e.g., murines, simians, equines, bovines, porcines, canines, felines, and the like), and most preferably includes humans. The term “individual” is not limited to a particular gender or age.

As used herein, the term “conditions involving aberrant erythrocyte potassium levels” refers to any condition characterized by abnormally high or low erythrocyte potassium levels. Examples of such conditions include, but are not limited to, hypertension, hyponatremia, and hypematremia.

As used herein the term “processor” or “computer processor” refers to a device that is able to read a program from a computer memory (e.g., ROM or other computer memory) and perform a set of steps according to the program. Processor may include non-algorithmic signal processing components (e.g., for analog signal processing).

As used herein, the term “algorithm” refers to a procedure devised to perform a function.

As used herein, the term “software” refers to any form of programmed machine-readable language or instructions (e.g., object code) that, when, loaded or otherwise installed, provides operating instructions to a device capable of reading those instructions, such as a computer or reader. Software useful in the present invention can be stored or reside on, as well as be loaded or installed from, one or more floppy disks, CD ROM disks, hard disks or any other form of suitable non-volatile electronic storage media. Software useful in the present invention can also be installed by downloading or other form of remote transmission, such as by using Local or Wide Area Network (LAN or WAN)-based, Internet-based, web-based or other remote downloading or transmission methods.

As used herein, the terms “computer memory” and “computer memory device” refer to any storage media readable by a computer processor. Examples of computer memory include, but are not limited to, RAM, ROM, computer chips, digital video disc (DVDs), compact discs (CDs), hard disk drives (HDD), and magnetic tape.

As used herein, the term “computer readable medium” refers to any device or system for storing and providing information (e.g., data and instructions) to a computer processor. Examples of computer readable media include, but are not limited to, DVDs, CDs, hard disk drives, magnetic tape, flash memory, and servers for streaming media over networks.

### DETAILED DESCRIPTION

Potassium (K⁺) in vascular smooth muscle cells (VSMC) and in the endothelium is a very important factor in vasodilatation. In VSMC, K⁺ efflux through Ca²⁺-activated K⁺ channels (K_{Ca}) causes membrane potential hyperpolarization and closes voltage-dependent Ca²⁺ channels, which leads to vasodilatation (see, e.g., Jaggar, J. H., et al., 1998 Acta Physiol Scand 164:577-587; herein incorporated by reference in its entirety). In the endothelium, K⁺ efflux, through intermediate conductance K_{ca} (IK_{Ca}), hyperpolarize VSMC via electrical coupling between endothelium and VSMC (see, e.g., Sandow, S. L. & Hill, C. E., 2000, Cir Res 86:341-346; herein incorporated by reference in its entirety), producing hyperpolarization and closing voltage-dependent Ca²⁺ channels. Stimulation of endothelial cells results in an outwardly rectifying K⁺ current (see, e.g., Coleman, H. A., et al., 2001, J. Physiol. 531:359-373; herein incorporated by reference in its entirety) and the combination of the K⁺ channel inhibitors apamin and charybdotoxin completely prevents the hyperpolarizing and vasodilating action of endothelium-derived hyperpolarizing factor (EDHF) in the rat hepatic artery (see, e.g., Anderson, A. J., et al., Br. J. Pharmacol. 95:1329-1335; herein incorporated by reference in its entirety). This observation indicates that K_{Ca} with the pharmacological characteristics of small and intermediate conductance K_{Ca} are involved in the EDHF vasodilatation.

In red blood cells (RBC), K⁺ efflux is also affected by IK_{Ca} (see, e.g., Del Carlo, B., et al., 2002, Biochim Biophys Acta, 558:133-141; Brugnara C, et al., 1993, J Biol Chem 268: 8760-8768; each herein incorporated by reference in their entireties). Ciclazindol, which abolishes EDHF relaxation in the presence of apamin, inhibits K_{Ca} in RBC (see, e.g., Anderson, A. J., et al., Br. J. Pharmacol. 95:1329-1335; herein incorporated by reference in its entirety) and cromakalim, a known K⁺ channel activator with vasodilator properties, opens K_{Ca} in RBC (see, e.g., Lijnen, P., et al., 1989, J. Hypertens. 7:403-407; herein incorporated by reference in its entirety). In addition, increased K⁺ efflux occurs in RBC treated with 8Br-cGMP, for example, by activation of I K_{Ca} as it occurs in VSMC (see, e.g., Price, J. M., et al., 1997, Life Sci. 61:1185-1192; herein incorporated by reference in its entirety). As such, K⁺ changes occurring in RBC are reflective of K⁺ changes in the VSMC or in the endothelium (see, e.g., Delgado, M. C. & Delgado-Almeida, A., 2003, J. Human Hypertens. 17:313-318; herein incorporated by reference in its entirety).

In experiments conducted during the course of the present invention, a relationship between erythrocyte potassium levels and blood pressure levels was identified. In particular, it was shown that individuals suffering from hypertension tend to have lower erythrocyte potassium levels than individuals not suffering from hypertension. The present invention provides a systematic approach that allows health care providers (e.g., physicians) to use erythrocyte potassium measurements as an indicator of hypertension or risk of developing hypertension. In particular, the present invention provides systems and methods for treating conditions involving erythrocyte potassium levels (e.g., hypertension), preventing the onset of hypertension, identifying individuals at risk for developing hypertension, and evaluating the effectiveness of treatments for conditions involving erythrocyte potassium levels (e.g., hypertension).

The present invention is not limited to a particular manner of obtaining erythrocyte potassium levels for an individual. In some embodiments, erythrocyte potassium levels are obtained through collection of an individual's blood. The present invention is not limited to a particular manner of collecting an individual's blood. In some embodiments, an individual's blood is collected through standard venipuncture techniques. In some embodiments, a blood collection vessel is used to collect an individual's blood. The present invention is not limited to a particular type or kind of blood collection vessel. In some embodiments, the blood collection vessel is a vacuum tube (e.g., a heparinized vacuum tube). In some embodiments, the blood collection vessel is a lancet configured to collect blood from an extremity (e.g., a finger tip). In some embodiments, the blood collection vessel is a standard syringe of any desired size. In preferred embodiments, the blood collection vessel is configured to obtain a blood sample from an individual for purposes of measuring erythrocyte potassium levels within the blood sample.

The present invention is not limited to a particular manner of measuring erythrocyte potassium levels for a blood sample. In some embodiments, standard laboratory techniques are used to measure erythrocyte potassium levels for a blood sample.

In some embodiments, a device (e.g., hand-held device, desktop device) is used to measure erythrocyte potassium levels for a blood sample. The device is not limited to a particular procedure or method for measuring erythrocyte potassium levels for a blood sample. In some embodiments, the device is configured to receive a blood sample having erythrocytes. The device is not limited to a particular manner of receiving a blood sample having erythrocytes. In some embodiments, the device is configured to separate the erythrocytes from a received blood sample. The device is not limited to a particular manner of separating erythrocytes from a received blood sample. In some embodiments, the device is configured to separate erythrocytes from a received blood sample through use of a cellulose strip coated with an erythrocyte-specific antibody. In some embodiments, the device is configured to separate erythrocytes from a cellulose strip coated with an erythrocyte-specific antibody. The device is not limited to a particular manner of separating erythrocytes from a cellulose strip coated with an erythrocyte-specific antibody. In some embodiments, the device is configured to separate erythrocytes from a cellulose strip coated with an erythrocyte-specific antibody through use of a semi-permeable membrane (e.g., cellulose semi-permeable membrane; synthetic semi-permeable membrane). The device is not limited to a particular type or kind of semi-permeable membrane. In some embodiments, the device is configured to hemolyze erythrocytes separated from a received blood sample. The device is not limited to a particular manner of hemolyzing erythrocytes separated from a received blood sample. In some embodiments, the device is configured to hemolyze erythrocytes separated from a blood sample through disruption of the erythrocyte membrane with distilled water. In some embodiment, the device is configured to measure an erythrocyte potassium level from a blood sample. The device is not limited to a particular manner of measuring an erythrocyte potassium level from a blood sample (e.g., flame photometry techniques; techniques utilizing potassium selective electrodes; etc.). In some embodiments, the devices are designed to measure an erythrocyte potassium level with imaging agents (e.g., bioluminescence, fluorescence, etc.).

In some embodiments, a desktop device is used to measure erythrocyte potassium levels for a blood sample. The present invention is not limited to a particular type of desktop device for measuring erythrocyte potassium levels in a blood sample. In some embodiments, the desktop device is configured to utilize techniques involving flame emission spectroscopy for purposes of measuring an erythrocyte potassium level in a blood sample. In some embodiments, the desktop device is configured to measure an erythrocyte potassium level with imaging agents (e.g., bioluminescence, fluorescence, etc.). In some embodiments, the desktop device is configured to utilize techniques involving potassium selective electrodes for purposes of measuring an erythrocyte potassium level in a blood sample.

In some embodiments, a hand-held device is used to measure erythrocyte potassium levels for a blood sample. The hand-held device is not limited to a particular size or weight. In some embodiments, the hand-held device is small enough to fit in a pocket (e.g., laboratory coat pocket, pants pockets, etc.). In some embodiments, the weight of the hand-held device is less than 5 pounds. In some embodiments, the hand-held device is configured to contact a blood sample, measure the erythrocyte blood levels within the sample, generate information regarding the measured erythrocyte blood levels, and communicate the generated information.

The hand-held device is not limited to a particular manner of contacting a blood sample. In some embodiments, the hand-held device has an applicator for contacting a blood sample. In some embodiments, the hand-held device has a reservoir for receiving a blood sample. In some embodiments, the hand-held device is configured to receive strips (e.g., cellulose strips) having thereon a blood sample. In some embodiments, the hand-held device is configured contact a blood sample such that a measurement of the erythrocyte potassium level of the blood sample can be calculated.

The hand-held device is not limited to a particular manner of measuring the erythrocyte potassium level for a contacted blood sample. In some embodiments, the hand-held device is configured to utilize techniques involving flame emission spectroscopy for purposes of measuring the erythrocyte potassium levels for a contacted blood sample. In some embodiments, the hand-held device is configured to utilize techniques involving potassium selective electrodes for purposes of measuring the erythrocyte potassium levels for a contacted blood sample. In some embodiments, the devices are designed to measure an erythrocyte potassium level with imaging agents (e.g., bioluminescence, fluorescence, etc.). In some embodiments, the hand-held device is configured to utilize standard laboratory techniques for purposes of measuring the erythrocyte potassium levels for a contacted blood sample. In some embodiments, the measured erythrocyte potassium levels are used to generate information.

The hand-held device is not limited to particular type or amount of generated information regarding the measured erythrocyte potassium levels. In some embodiments, the hand-held device is configured to generate an individual's risk for hypertension or a related condition. In some embodiments, the hand-held device is configured to generate the change in measured erythrocyte potassium levels over a desired period of time (e.g., the length of a particular treatment). In some embodiments, the hand-held device is configured to generate a series of measured erythrocyte potassium levels from a plurality of time points.

The hand-held device is not limited to a particular manner of generating information regarding the measured erythrocyte potassium levels. In some embodiments, the hand-held device has therein a processor configured to generate information regarding the measured erythrocyte potassium levels. The present invention is not limited to a particular type of processor. In some embodiments, the processor is designed to generate information regarding a measured erythrocyte potassium level and communicate the information. In some embodiments, the hand-held device has therein a memory of sufficient size to store large amounts of data (e.g., information regarding measured erythrocyte potassium levels). In some embodiments, the memory is at least 100 Mb (e.g., 150 Mb, 700 Mb, 1 gig, 100 gigs, 1 terabyte, 100 terabytes). In some embodiments, the hand-held device has therein software configured to interact with the processor. In some embodiments, the software is configured to generate information regarding the measured erythrocyte potassium levels.

The present invention is not limited to a particular type of software. In some embodiments, the software is designed to compare the measured erythrocyte potassium level with a predetermined erythrocyte potassium level threshold (e.g., approximately 90 mmol/L per cell for adult individuals and approximately 94 mmol/L per cell for child individuals). The software is not limited to a particular manner of comparing the measured erythrocyte potassium level and the predetermined erythrocyte potassium level threshold. In some embodiments, the software is configured to label a measured erythrocyte potassium level as low risk for developing hypertension or related condition if the measured erythrocyte potassium levels are above the predetermined erythrocyte potassium level threshold. In some embodiments, the software is configured to label a measured erythrocyte potassium level as high risk for developing hypertension or related condition if the measured erythrocyte potassium levels are at or below the predetermined erythrocyte potassium level threshold. In some embodiments, the software is programmable to include additional information in the comparison of measured erythrocyte potassium levels and the predetermined erythrocyte potassium level threshold (e.g., genetic information about an individual, medication being used by an individual, length of treatment, family history, etc.). In some embodiments, the software is configured to generate comparison information regarding a plurality of measured erythrocyte potassium levels over any desired amount of time.

The hand-held device is not limited to a particular manner of communicating the generated information to a user. In some embodiments, the hand-held device has a display monitor for displaying the generated information (e.g., textually based messages, color based messages). In some embodiments, the hand-held device has an audio system (e.g., a speaker system) for audibly presenting the generated information. In some embodiments, the hand-held device is configured to provide the generated information to a health care management system (e.g., network) through wireless communication. In some embodiments, the hand-held device is configured to provide the generated information to an Internet based location (e.g., website) through wireless communication.

In some embodiments, the devices have therein a processor for obtaining an individual's erythrocyte potassium and/or monitoring an individual's hypertension risk. In some embodiments, the devices are configured to interact (e.g., wireless) with a database containing information for a patient (e.g., a hospital database). In some embodiments, the devices have a memory for storing generated information (e.g., an individual's potassium levels at different times) over a period of time. In some embodiments, the memory is at least 100 Mb (e.g., 150 Mb, 700 Mb, 1 gig, 100 gigs, 1 terabyte, 100 terabytes). In some embodiments, the interacting is wireless communication to the Internet.

In some embodiments, the present invention provides a kit for obtaining an individual's erythrocyte potassium level. The present invention is not limited to particular parts of the kit. In some embodiments, the kit comprises a blood collection vessel and a device for measuring erythrocyte potassium levels. In some embodiments, the device is a hand-held device. In some embodiments, the device is a desktop device.

In some embodiments, the present invention provides kits for measuring an individual's erythrocyte potassium level from a blood sample. The present invention is not limited to particular parts of the kit. In some embodiments, the kit comprises a blood collection vessel and a device for measuring erythrocyte potassium levels from a blood sample. In some embodiments, the device is a hand-held device. In some embodiments, the device is a desktop device. In some embodiments, the blood collection vessel with a lancet with a cellulose strip having thereon antibodies for red blood cells.

In some embodiments, the present invention provides home test kits for individual use. In some embodiments, the home test kit comprises a hand-held device for measuring an erythrocyte potassium level from a bodily sample (e.g., blood sample) and instructions for operating the hand-held device. In some embodiments, the home test kit comprises test strips for measuring an erythrocyte potassium level from a bodily sample (e.g., blood sample) and instructions for operating the test strips. In some embodiments, the test strips and hand-held device are configured to display a user's risk for hypertension. In some embodiments, the test strips and hand-held device are configured to display a numerical based erythrocyte potassium level value. In some embodiments, the home test kits comprise instructions for interpreting displayed results.

In experiments conducted during the course of the present invention demonstrate a quantitative relationship between an individual's (e.g., adults, children) erythrocyte potassium level and hypertension. As such, the present invention provides a novel standardized range of erythrocyte potassium levels distinguishing hypertensive individuals (e.g., adult, children) and individuals (e.g., adults, children) identified as being at high risk for developing hypertension from non-hypertensive individuals (e.g., adults, children) and individuals (e.g., adults, children) identified as being at low risk for hypertension. In addition, the present invention shows that maintaining erythrocyte potassium levels within or above a desired threshold provides a means for monitoring and managing a particular treatment.

In experiments conducted during the course of the present invention, a relationship between hypertension risk and erythrocyte potassium levels was identified. In particular, it was shown that individuals having erythrocyte potassium levels above a certain threshold (hereinafter, “erythrocyte potassium level threshold”) are at low risk for hypertension, while individuals having erythrocyte potassium levels below the erythrocyte potassium level threshold are at high risk for hypertension. The erythrocyte potassium level threshold is not limited to a particular value or range of values. For adult human individuals, the identified erythrocyte potassium level threshold was approximately 90 mmol/L per cell. For child human individuals, the identified erythrocyte potassium level threshold was approximately 94 mmol/L per cell.

The identified erythrocyte potassium level threshold values are not limited to a particular value or range of values. In some embodiments, “approximately 90 mmol/L per cell” describes erythrocyte potassium levels between 89 mmol/L per cell and 91 mmol/L per cell. In some embodiments, “approximately 94 mmol/L per cell” describes erythrocyte potassium levels between 93 mmol/L per cell and 95 mmol/L per cell.

In some embodiments, the erythrocyte potassium level threshold includes erythrocyte potassium level values higher or lower than “approximately 90 mmol/L per cell” (e.g., . . . 80 mmol/L per cell, 86 mmol/L per cell, 88 mmol/L per cell, 92 mmol/L per cell, 94 mmol/L per cell, 100 mmol/L per cell . . . ) and “approximately 94 mmol/L per cell” (e.g., . . . 84 mmol/L per cell, 90 mmol/L per cell, 92 mmol/L per cell, 96 mmol/L per cell, 98 mmol/L per cell, 104 mmol/L per cell . . . ). In some embodiments, the erythrocyte potassium level threshold varies depending on other criteria (e.g., an individual's related medical condition, an individual's genetic profile, etc.).

The present invention provides systems, methods, kits and devices that utilize the identified erythrocyte potassium level threshold in, for example, the monitoring, treatment, prevention, evaluation, and diagnosis of hypertension and risk of hypertension and related conditions.

In some embodiments, the prevent invention provides a novel method of diagnosing an individual's (e.g., adult or child) risk for hypertension. The method of diagnosing is not limited to a particular method. In some embodiments, the method of diagnosing comprises an analysis of the individual's erythrocyte potassium level. In such embodiments, individuals having an erythrocyte potassium level above the erythrocyte potassium level threshold are diagnosed as being at low risk for hypertension, while individuals having an erythrocyte potassium level below the erythrocyte potassium level threshold are diagnosed as being at high risk. The method of diagnosing is not limited to a particular age distinction between children and adults. In some embodiments, children are considered to be younger than twenty-one years of age (e.g., 20, 19, 18, 17 . . . ). In some embodiments, the method comprises additional steps including, but not limited to, providing additional diagnostic tests, and providing various forms of treatment.

In some embodiments, the present invention provides a method of monitoring the effectiveness of a particular treatment or combination of treatments. In some embodiments, the method of monitoring comprises evaluating the effectiveness of a treatment through monitoring the change in erythrocyte potassium levels for an individual over a period of time (e.g., the course of the treatment) or maintaining erythrocyte potassium levels above a desired threshold over the course of treatment. The method of monitoring is not limited to a particular series of steps. In some embodiments, the method of monitoring comprises measurement of an individual's erythrocyte potassium level, administration of a treatment, measuring the individual's erythrocyte potassium levels during the course of the treatment, and evaluating the effectiveness of the treatment based on the individual's erythrocyte potassium levels during the course of the treatment. In some embodiments, the first measurement of the individual's erythrocyte potassium level occurs after the onset of treatment. In some embodiments, the method of monitoring further comprises the step of changing a treatment (e.g., continuing a treatment, stopping a treatment, starting a new treatment, increasing a treatment, decreasing a treatment, etc.) based upon the evaluated effectiveness of an administered treatment. In some embodiments, the monitoring of the individual's erythrocyte potassium levels is conducted at a plurality of time points (e.g., weekly, daily, hourly, continuously).

In some embodiments, a treatment is considered effective when an individual's erythrocyte potassium levels increase over the course of the treatment or the erythrocyte potassium levels are maintained at a desired level over the course of the treatment. In some embodiments, a treatment is considered ineffective when an individual's erythrocyte potassium levels decrease over the course of a treatment or the erythrocyte potassium levels are maintained at an undesired level over the course of the treatment.

In some embodiments, the effectiveness of a treatment is monitored in comparison to an identified erythrocyte potassium level threshold. In such embodiments, a treatment is considered effective when over the course of the treatment an individual's erythrocyte potassium levels remain above the erythrocyte potassium level threshold. In some embodiments, a treatment is considered ineffective when over the course of the treatment an individual's erythrocyte potassium levels remain at or below the erythrocyte potassium level threshold.

In some embodiments, in addition to monitoring an individual's erythrocyte potassium levels over a period of time, the method of monitoring involves additional steps directed toward evaluating the effectiveness of a treatment. The present invention is not limited to particular steps directed toward evaluating the effectiveness of a treatment. In some embodiments, the additional steps include, but are not limited to, analysis of CBC count, analysis of serum electrolytes, analysis of serum creatinine, analysis of serum glucose, analysis of uric acid, urinalysis, a lipid profile analysis (e.g., total cholesterol, low-density lipoprotein (LDL) and high-density lipoprotein (HDL), and triglycerides), imaging tests (e.g., electrocardiography), electrocardiograms, obtaining total body potassium measurements by bio-impedance, analysis of 12-hour urinary potassium excretion, obtaining serial plasma glucose and insulin levels, and ambulatory blood pressure monitoring.

The present invention is not limited to a particular type of treatment or combination of treatments (e.g., preventing the onset of hypertension in individuals identified as being at high or low risk for developing hypertension; treating individuals diagnosed as having hypertension; experimental treatment). In some embodiments, the treatment includes a life-style change, an experimental treatment, and/or a pharmacological treatment.

The present invention is not limited to a particular type of life-style change. In some embodiments, the life-style change comprises a dietary change (e.g., reduced intake of dietary saturated fat and cholesterol). In some embodiments, the life-style change involves a reduction in alcohol intake (e.g., limiting alcohol intake to no more than 1 oz (30 mL) of ethanol (e.g., 24 oz (720 mL) of beer, 10 oz (300 mL) of wine, 2 oz (60 mL) of 100-proof whiskey) per day or 0.5 (15 mL) ethanol per day for women and people of lighter weight). In some embodiments, the life-style change involves an increase in aerobic activity for the individual (e.g., increasing aerobic activity to 30-45 minutes most days of the week). In some embodiments, the life-style change involves reduction or elimination of nicotine intake (e.g., stopping smoking). In some embodiments, the life-style change involves adequate intake of dietary calcium and magnesium. In some embodiments, the life-style change involves reduction of sodium intake to no more than 100 mmol/d (2.4 g sodium or 6 g sodium chloride). In some embodiments, the life-style change comprises a combination of life-style changes.

The present invention is not limited to a particular type of pharmacological treatment. In some embodiments, the pharmacological treatment comprises one or more pharmacological treatments comprising administration of one or more drugs including, but not limited to, hydrochlorothiazide, spironolactone, amiloride, furosemide, prazosin, atenolol, metoprolol, propranolol, labetalol, carvedilol, hydralazine, minoxidil, diltiazem, verapamil, nifedipine, captopril, enalapril, lisinopril, ramipril, losartan, valsartan, eprosartan, olmesartan, eplerenone, methyldopa, clonidine. In some embodiments, the pharmacological treatment comprises the administration of one or more potassium sparing drugs. The present invention is not limited to a particular kind of potassium sparing drug. In some embodiments, the potassium sparing drug is amiloride and triamterene. In some embodiments, the pharmacological treatment comprises administration of one or more aldosterone blocking drugs. The present invention is not limited to a particular kind of aldosterone blocking drugs. In some embodiments, the aldosterone blocking drug is spironolactone or eplerenone. In some embodiments, the pharmacological treatment comprises administration of one or more potassium oral supplements. The present invention is not limited to a particular kind of potassium oral supplement. In some embodiments, the potassium oral supplement is any form of potassium chloride (e.g., oral capsule extended release, oral powder for solution, oral powder for suspension-extended release, oral solution, oral tablet, oral tablet-extended release, and sublingual tablet). In some embodiments, the pharmacological treatment comprises administration of an anti-hypertensive drug. The present is not limited to a particular kind of anti-hypertensive drug. In some embodiments, the anti-hypertensive drug is a thiazide diuretic, a loop diuretic, a beta blocker, a beta blocker with intrinsic sympathomimetic activity, a combined alpha and beta blocker, an angiotensin converting enzyme inhibitor, an angiotensin II antagonist, a calcium channel blocker, an alpha-1 blocker, a central alpha-2 agonist, or a direct vasodilator. In some embodiments, the pharmacological treatment comprises administration of a combination of drugs.

The present invention is not limited to a particular type of experimental treatment. In some embodiments, the experimental treatment comprises administration of a new pharmacological agent (e.g., a new medication). In some embodiments, the experimental treatment comprises an experimental procedure involving stem cells (e.g., stem cell therapy). In some embodiments, the experimental treatment comprises an experimental procedure involving cellular therapy (e.g., therapies involving mononuclear transformed cells). In some embodiments, the experimental treatment comprises an experimental procedure involving gene therapy (e.g., gene therapy directed to correct a defect in an individual's erythrocyte potassium level pathways and related pathways (e.g., erythrocyte transport pathways)). In some embodiments, the experimental treatment comprises an experimental surgical procedure. This aspect of the present invention permits the screening of new pharmacological agents, new cellular therapies, new forms of gene therapy, new surgical procedures, new life-style modifications, and other new forms of treatment, and combinations thereof, for an ability to modify an individual's erythrocyte potassium levels.

The present invention provides methods of treating conditions involving aberrant erythrocyte potassium levels (e.g., hypertension, hyponatremia, hypernatremia, insulin resistance). The present invention is not limited to a particular condition involving aberrant (e.g., too high, too low) erythrocyte potassium levels. In some embodiments, the condition involving aberrant erythrocyte potassium levels is hypertension. In some embodiments, the condition involving aberrant erythrocyte potassium levels is a metabolic syndrome, including, but not limited to hypertension, dyslipidemia, hyperglycemia and coronary artery disease. In some embodiments, the condition involving aberrant erythrocyte potassium levels is type 2 diabetes mellitus.

The present invention is not limited to a particular method of treating conditions involving aberrant erythrocyte potassium levels (e.g., hypertension, insulin resistance). In some embodiments, the method involves the administration of a treatment (e.g., a life-style modification, a pharmacological treatment, an experimental treatment) to an individual having or suspected of having a condition involving aberrant erythrocyte potassium levels (e.g., hypertension) and the subsequent monitoring of the treatment through measurement of the individual's erythrocyte potassium levels. The present invention is not limited to a particular type of treatment. In some embodiments, the treatment is designed to modify (e.g., raise or lower) the individual's erythrocyte potassium levels.

In some embodiments, the treatment is designed to prevent the onset of a condition involving aberrant erythrocyte potassium levels (e.g., hypertension, insulin resistance). In such embodiments, an individual is administered a treatment designed to increase the individual's erythrocyte potassium levels or maintain the individual's erythrocyte potassium levels above the erythrocyte potassium level threshold.

In some embodiments, the treatment is designed to improve insulin sensitivity in individuals suffering from insulin resistance. In such embodiments, an individual is administered a treatment designed to increase the individual's erythrocyte potassium levels or maintain the individual's erythrocyte potassium levels above the erythrocyte potassium level threshold.

In some embodiments, the present invention provides drug screening assays (e.g. to screen for new drugs for treating conditions involving aberrant erythrocyte potassium levels (e.g., hypertension, insulin resistance). The screening methods of the present invention utilize the methods for measuring an individual's erythrocyte potassium levels provided in the present invention. For example, in some embodiments, the present invention provides methods of screening for compounds that alter (e.g., increase or decrease), directly or indirectly, erythrocyte potassium levels. In some embodiments, candidate compounds are antisense agents (e.g., siRNAs, oligonucleotides, etc.) directed against pathways associated with erythrocyte potassium levels.

### EXAMPLES

### Example 1

This example describes the subjects used in the experiments used during the course of the present invention. The study was performed on 50 patients (26 males and 24 females) with untreated essential hypertension, 32 of their offspring (13 males and 19 females) and 50 age- and sex-matched controls (26 males and 24 females). All subjects had a normal dietary salt intake and all subjects gave a written informed consent. Patients were recruited at the Clinical Research Unit, University of Carabobo, Venezuela. Exclusion criteria were any secondary form of hypertension, diabetes mellitus, renal insufficiency, gastrointestinal disorders, pregnancy or other significant medical conditions. Patients taking drugs that could affect blood pressure (BP) were also excluded. In all, 41 patients were never treated for hypertension. Six patients were treated with ace inhibitors, two with β-blockers and one with calcium channel blocker. All of them had a 2-week washout before starting the study and those receiving β-blockers had dosage tapering before the 2-week washout. All patients underwent physical examination and routine biochemical tests before the study.

Offspring of hypertensives were recruited through their parents. A total of 14 hypertensive parents (10 mothers and four fathers) representing 14 families were studied. All of the spouses were normotensive and no laboratory studies were performed in them. Offspring 12 years old and older were invited to participate independent of their blood pressure level.

The normotensive controls had systolic SBP less than 140 mmHg and diastolic DBP less than 90 mmHg and were matched with hypertensive patients by sex and age. They had no history of serious disease and had taken no medication for at least 6 weeks. A subgroup of the normotensive controls (n=10) that matched hypertensive patients by age and body mass index (BMI) was selected to exclude the influence of overweight.

### Example 2

This example describes the blood pressure and other physical measure procedures used in the experiments conducted during the course of the present invention. All subjects had their BP measured using a mercury sphygmomanometer on two separate occasions in the morning. Subjects rested seated for 5 min, after which blood pressure recordings were done in triplicate (each reading separated by 2 min using the appropriate cuff size based on the upper midarm. Blood pressure values were the mean of three recordings at the second visit. Subjects were defined as having high BP if either SBP was ≧140 mmHg or DBP (Korotkoff phase V) was ≧90 mmHg. Subjects were defined as normotensive if systolic blood pressure was ≦140 mmHg and diastolic blood pressure was ≦90 mmHg. Height was measured using a wall-mounted tape measure and weight determined on a balance-beam scale.

### Example 3

This example describes the biochemical measurement procedures used in the experiments conducted during the course of the present invention. On the day of the study, subjects provided a 12-hour urine sample after an overnight fast. Peripheral venous blood was drawn, immediately centrifuged and the buffy coat discarded. RBC_{Ki} and RBC_{Nai} were measured in the supernatants of lysed RBC. For RBC_{Ki} 100 ml of packed RBC was lysed in 10 ml of distilled water. For RBC_{Nai}, RBC were washed in isotonic choline solution ×3 and then lysed in 10 ml of distilled water. After appropriate dilutions, RBC_{Ki} and RBC_{Nai} were measured in duplicate in their respective hemolysates by flame photometry (Corning 410C, Cambridge, Mass., USA) using a K⁺/Na⁺ standard of 100/10 mmol/l. Values were expressed as mmol/l. The coefficients of variation for RBC_{Ki} and RBC_{Nai} were <3%. In 20 randomly selected samples, the amount of trapped plasma was measured with ³H inulin (0.870.07% for 100 ml of RBC). Na⁺ and K⁺, in both plasma and urine, were measured by a flame photometer. Ionized Ca2⁺ was measured directly in fresh plasma using a Ciba Corning 634 Ca2⁺/pH analyzer (Medfield, Mass., USA). The concentrations of fasting plasma glucose, triglycerides and cholesterol were measured by standard methods in hypertensives and normotensive controls.

### Example 4

This example describes the statistical analysis used in the experiments conducted during the course of the present invention. All statistical procedures were performed using the SAS system for Windows release 6.12. Results are expressed as mean±s.e. Statistical evaluations was by analysis of variance. To reduce the probability of significant differences arising by chance, Bonferroni's correction was applied following analysis of variance. Differences were considered significant when P<0.05. The relation between RBC_{Ki} and other variables was evaluated using the Pearson partial correlation coefficient (age and gender as partial correlation variables). A subgroup of hypertensives and age- and BMI-matched normotensive controls was evaluated in order to exclude the effect of overweight.

### Example 5

The characteristics of hypertensives, offspring of hypertensives and normotensive controls are shown in Table 1. There were no significant differences among the three groups in gender distribution, RBC_{Nai}, plasma Na⁺ or 12-h urinary Na⁺:K⁺ ratio. SBP was significantly higher in hypertensives than in offspring of hypertensives and in normotensive controls. In spite of the fact that the offspring of hypertensives were younger than normotensive controls, their average SBP (but not DBP) was significantly higher.

TABLE 1

| | Hypertensives | Offspring | Controls | HT vs OF | HT vs CO | OF vs CO |
|---|---|---|---|---|---|---|
| Male/female | 26/24 | 13/19 | 26/24 | NS | NS | NS* |
| Age (years) | 45 ± 1 | 22 ± 2 | 45 ± 1 | P < 0.001 | NS | P < 0.001 |
| Body mass index (kg/m²) | 27 ± 1 | 25 ± 1 | 25 ± 1 | P < 0.05 | P < 0.01 | NS |
| Systolic BP (mmHg) | 156 ± 2 | 134 ± 3 | 121 ± 2 | P < 0.001 | P < 0.001 | P < 0.001 |
| Diastolic BP (mmHg) | 103 ± 1 | 76 ± 2 | 74 ± 1 | P < 0.001 | P < 0.001 | NS |
| RBC_{Ki} (mmol/l cell) | 81.4 ± 0.7 | 85.8 ± 1.2 | 93.1 ± 0.7 | P < 0.01 | P < 0.001 | P < 0.001 |
| RBC_{Nal} (mmol/l cell) | 6.75 ± 0.16 | 7.25 ± 0.31 | 6.79 ± 0.17 | NS | NS | NS |
| Plasma K (mmol/l) | 3.65 ± 0.06 | 3.62 ± 0.10 | 3.99 ± 0.06 | NS | P < 0.001 | P < 0.01 |
| Plasma Na (mmol/l) | 139.4 ± 0.4 | 1.39 ± 0.8 | 139.4 ± 0.5 | NS | NS | NS |
| Ionized Ca²⁺ (mmol/l) | 1.07 ± 0.01 | 1.05 ± 0.02 | 1.09 ± 0.01 | NS | NS | NS |
| 12-h urinary Na:K ratio | 3.65 ± 0.31 | 3.28 ± 0.60 | 3.72 ± 0.38 | NS | NS | NS |

HT = hypertensives;
OF = offspring;
CO = controls;
BP = blood pressure;
RBC_{Ki} = red blood cell potassium;
RBC_{Nal} = red blood cell sodium.
Values represent the mean ± s.e.m.
Variables are adjusted for age and gender.
*χ² analysis.

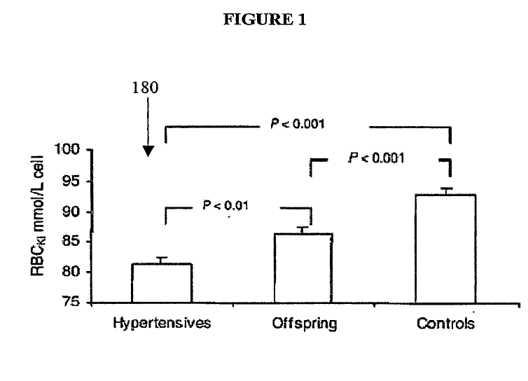

RBC_{Ki} was significantly lower in hypertensives than in offspring of hypertensives and in normotensive controls (see FIG. 1). Offspring of hypertensives had significantly lower RBC_{Ki} than normotensive controls. Plasma K⁺ was significantly lower both in hypertensives and offspring of hypertensives when compared to normotensive controls. No difference was observed between hypertensives and offspring of hypertensives. Ionized Ca²⁺ did not differ between hypertensives, offspring of hypertensives and in normotensive controls.

A significant negative correlation was found in hypertensives between RBC_{Ki} and DBP (r=−0.27, P>0.04) and in offspring of hypertensives between RBC_{Ki} and DBP (r=−0.43, P>0.02). A significant correlation was found in hypertensives between RBC_{Ki} and plasma K⁺ (r=−0.3, P>0.02). Hypertensives exhibit a trend of higher RBC_{Ki} levels with higher ionized Ca₂₊ (r=−0.2, P>0.1). In the three groups, no significant correlations were observed between RBC_{Nai} and SBP (r=−0.002, P>0.05) or DBP (r=−0.29, P>0.05); between Ca²⁺ and SBP (r=−0.17, P>0.05) or DBP (r=−0.02, P>0.05); between plasma K⁺ and SBP (r=−0.06, P>0.05) or DBP (r=−0.01, P>0.05); between plasma Na⁺ and SBP (r=−0.02, P>0.05) or DBP (r=−0.14, P>0.05); and between urinary Na⁺:K⁺ ratio and SBP (r=−0.04, P>0.05) or DBP (r=−0.03, P>0.05).

Hypertensives had significantly higher glucose (6.21±0.11 vs 4.33±0.11 mmol/l, P>0.001), triglycerides (2.10±0.68 vs 1.01±0.27 mmol/l, P=0.001) and cholesterol levels (6.27±0.42 vs 4.79±0.70 mmol/l, P=0.003) than age-sex-matched normotensive controls.

There were no significant differences in RBC_{Ki} (80.7±0.4 vs 81.170.4 mmol/l cell, P>0.05) or RBC_{Nai} (6.7370.14 vs 6.75±0.13, P>0.05) between never-treated hypertensives and those in whom the antihypertensive treatment was stopped 2 weeks prior to the study.

In the subgroup of hypertensives (n=10) and normotensive controls (n=10) matched by age (48±2 vs 48±2 years, P>0.05) and BMI (29±1 vs 29±1 kg/m², P>0.05), significant difference was observed in RBC_{Ki} (78.3±1.6 vs 91.3±1.6 mmol/l cell, P<0.0001). No significant differences were observed in RBC_{Nai}, (7.31±0.42 vs 6.51±0.45 mmol/l cell, P>0.05), plasma K⁺ (3.69±0.19 vs 4.18±0.19 mmol/l, P>0.05), plasma Na⁺ (139.5±0.8 vs 138.1±0.8 mmol/l, P>0.05), Na⁺:K⁺ ratio (3.25±0.56 vs 2.98±0.64 mmol/l, P>0.05) and ionized calcium (1.01±0.02 vs 1.01±0.02 mmol/l, P>0.05).

### Example 6

A mixture analysis (see, e.g., Hunt G, Chapman RE, 2001, Paleobiology 27(3):466-484; herein incorporated by reference in its entirety) was used to establish the likelihood of a bimodal distribution of erythrocyte potassium content. The method analyzes, for example, distributions that are hypothesized to be a mixture of sub-distributions. The program uses a maximum-likelihood (ML) approach to estimate the parameters of each sub-distribution. The hill-climbing routine that is used to find the ML estimates is an expectation-maximization (EM) algorithm. Any two solutions to the same model (for example, a 2-group model) are compared directly through their likelihood (or the natural logarithm of the likelihood, the log-likelihood).

Children Biomodality: In children, the analysis demonstrated that erythrocyte potassium fit significantly better in a bimodal than a unimodal or trimodal distribution. Bootstrap testing was used to test the likelihood that a bimodal distribution rejected a unimodal distribution (1 vs. 2 group models). Bootstrap testing showed an observed −2lambda: 10.1200 which was statistically significant (p=0.010 (bootstraps 990/1000)). Bootstrap testing was used to test the likelihood that a trimodal distribution rejected a bimodal distribution (2 vs. 3 group models). Bootstrap testing showed an observed −2lambda: 4.0900 which was not statistically significant (p=0.446 (bootstraps 554/1000)).

Children Values: The mixture analysis showed the mean and variance for low (group 1) and high (group 2) erythrocyte potassium groups (see document children-groups.txt) in the following manner:

Log-Likelihood: −308.0631

| Group | Mean | Variance | Mixing Proportion |
|---|---|---|---|
| 1 | 90.5195 | 15.1537951 | 0.8507 |
| 2 | 101.5858 | 15.1537951 | 0.1493 |

Upper value for low erythrocyte potassium in children (group 1): The upper value of group 1 is the cutoff point to establish what subject has “Low Erythrocyte Potassium Content” or “High Risk Potassium”. A value above the upper value is considered “High Erythrocyte Potassium Content” or “Low Risk Potassium”.

Group 1: upper value=94.4˜94 mmol/L cell (mean+square root of the variance).

Adult Bimodality: In adults, the analysis demonstrated that erythrocyte potassium fit significantly better a bimodal than a unimodal or trimodal distribution. Bootstrap testing was used to test the likelihood that a bimodal distribution rejected a unimodal distribution (1 vs. 2 group models). Bootstrap testing showed an observed −2lambda: 76.307 which was statistically significant (p=<0.0001 (bootstraps 0/200)). Bootstrap testing was used to test the likelihood that a trimodal distribution rejected a bimodal distribution (2 vs. 3 group models). Bootstrap testing showed an observed −2lambda: 1.38 which was not statistically significant (p=1 (bootstraps 0/200)).

Adult Values: The mixture analysis showed the mean and variance for low (group 1) and high (group 2) erythrocyte potassium groups (see document adult-groups.txt) in the following manner:

Log-Likelihood: −409.7647

| Group | Mean | Variance | Mixing Proportion |
|---|---|---|---|
| 1 | 90.0933 | 0.0192798 | 0.2033 |
| 2 | 92.4895 | 63.9386223 | 0.7967 |

Upper value for low erythrocyte potassium in adults (group 1): The upper value of group 1 is the cutoff point to establish what subject has “Low Erythrocyte Potassium Content” or “High Risk Potassium”. A value above the upper value is considered “High Erythrocyte Potassium Content” or “Low Risk Potassium”.

Group 1: upper value=90.2˜90 mmol/L cell (mean+square root of the variance).

All publications and patents mentioned in the above specification are herein incorporated by reference. Various modifications and variations of the described method and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention that are obvious to those skilled in the relevant fields are intended to be within the scope of the following claims.

We claim:

1. A method of evaluating the effectiveness of a hypertension treatment for an individual, comprising:
a) administering said hypertension treatment to said individual,
b) obtaining erythrocyte potassium levels for said individual, and
c) evaluating the effectiveness of said hypertension treatment based upon said obtained erythrocyte potassium levels.

2. The method of claim 1, further comprising the step of obtaining a baseline erythrocyte potassium level for said individual prior to said administering of said hypertension treatment.

3. The method of claim 1, wherein said obtaining erythrocyte potassium levels occurs during the course of said hypertension treatment.

4. The method of claim 1, further comprising the step of obtaining a post-treatment erythrocyte potassium level.

5. The method of claim 1, further comprising the step of adjusting or monitoring said treatment so as to maintain said erythrocyte potassium level at or above a desired erythrocyte potassium level.

6. The method of claim 5, wherein said individual is a human being over 20 years of age.

7. The method of claim 6, wherein said desired erythrocyte potassium level is approximately 90 mmol/L per cell.

8. The method of claim 6, wherein said approximately 90 mmol/L per cell is 90 mmol/L per cell.

9. The method of claim 5, wherein said individual is a human being under 21 years of age.

10. The method of claim 9, wherein said desired erythrocyte potassium level is approximately 94 mmol/L per cell.

11. The method of claim 10, wherein said approximately 94 mmol/L per cell is 94 mmol/L per cell.

12. The method of claim 6, wherein an increase in said individual's erythrocyte potassium levels above approximately 90 mmol/L per cell during said hypertension treatment administration indicates said hypertension treatment is effective.

13. The method of claim 6, wherein a decrease in said individual's erythrocyte potassium levels below approximately 90 mmol/L per cell during said hypertension treatment administration indicates said hypertension treatment is ineffective.

14. The method of claim 9, wherein an increase in said individual's erythrocyte potassium levels above approximately 94 mmol/L per cell during said hypertension treatment administration indicates said hypertension treatment is effective.

15. The method of claim 9, wherein a decrease in said individual's erythrocyte potassium levels below approximately 94 mmol/L per cell during said hypertension treatment administration indicates said hypertension treatment is ineffective.

16. The method of claim 1, wherein said obtaining erythrocyte potassium levels comprises a collection of a blood sample from said individual and analysis of said blood sample.

17. The method of claim 16, wherein said analysis of said blood sample comprises measurement of said erythrocyte potassium levels.

18. The method of claim 16, wherein said measurement is accomplished with a hand-held device.

19. The method of claim 1, wherein said hypertension treatment comprises life-style modification.

20. The method of claim 1, wherein said hypertension treatment comprises a pharmacological treatment.

21. The method of claim 1, wherein said hypertension treatment comprises an experimental treatment.

22. The method of claim 19, wherein said life-style modification comprises one or more life-style modifications selected from the group consisting of a dietary change, a reduction in alcohol intake, an increase in aerobic activity, a reduction or elimination of nicotine intake, an adequate intake of dietary calcium and magnesium, and a reduction of sodium intake.

23. The method of claim 20, wherein said pharmacological treatment comprises one or more pharmacological treatments selected from the group consisting of administration of one or more potassium sparing drugs, administration of one or more aldosterone blocking drugs, administration of one or more potassium oral supplements, and administration of one or more anti-hypertensive drugs.

24. A method of diagnosing risk for hypertension, comprising:
a) obtaining an adult individual's erythrocyte potassium level, and
b) diagnosing said individual as being:
i) at low risk for hypertension if said erythrocyte potassium level is above approximately 90 mmol/L per cell, or
ii) at high risk for hypertension if said erythrocyte potassium level is at or below approximately 90 mmol/L per cell.

25. The method of claim 24, wherein said approximately 90 mmol/L per cell is 90 mmol/L per cell.

26. The method of diagnosing of claim 24, further comprising the step of administering a hypertension treatment to said individual if said adult individual is diagnosed as being at high risk for hypertension.

27. The method of claim 26, wherein said hypertension treatment comprises a life-style modification.

28. The method of claim 26, wherein said hypertension treatment comprises a pharmacological treatment.

29. The method of claim 27, wherein said life-style modification comprises one or more life-style modifications selected from the group consisting of a dietary change, a reduction in alcohol intake, an increase in aerobic activity, a reduction or elimination of nicotine intake, an adequate intake of dietary calcium and magnesium, and a reduction of sodium intake.

30. The method of claim 28, wherein said pharmacological treatment comprises one or more pharmacological treatments selected from the group consisting of administration of one or more potassium sparing drugs, administration of one or more aldosterone blocking drugs, administration of one or more potassium oral supplements, and administration of one or more anti-hypertensive drugs.

31. The method of claim 24, wherein said adult individual is a human being over 20 years old.

32. A method of diagnosing risk for hypertension, comprising:
a) obtaining an child individual's erythrocyte potassium level, and
b) diagnosing said individual as being:
i) at low risk for hypertension if said erythrocyte potassium level is above approximately 94 mmol/L per cell, or
ii) at high risk for hypertension if said erythrocyte potassium level is at or below approximately 94 mmol/L per cell.

33. The method of claim 32, wherein said approximately 94 mmol/L per cell is 94 mmol/L per cell.

34. The method of diagnosing of claim 32, further comprising the step of administering a hypertension treatment to said individual if said adult individual is diagnosed as being at high risk for hypertension.

35. The method of claim 34, wherein said hypertension treatment comprises a life-style modification.

36. The method of claim 34, wherein said hypertension treatment comprises a pharmacological treatment.

37. The method of claim 35, wherein said life-style modification comprises one or more life-style modifications selected from the group consisting of a dietary change, a reduction in alcohol intake, an increase in aerobic activity, a reduction or elimination of nicotine intake, an adequate intake of dietary calcium and magnesium, and a reduction of sodium intake.

38. The method of claim 36, wherein said pharmacological treatment comprises one or more pharmacological treatments selected from the group consisting of administration of one or more potassium sparing drugs, administration of one or more aldosterone blocking drugs, administration of one or more potassium oral supplements, and administration of one or more anti-hypertensive drugs.

39. The method of claim 32, wherein said adult individual is a human being under 21 years old.

40. A method of treating hypertension, comprising
administering to an individual diagnosed as having hypertension a treatment configured to increase said individual's erythrocyte potassium level above a predetermined erythrocyte potassium level threshold,
obtaining at least one measurement of said individual's erythrocyte potassium levels during the course of said treatment, and
monitoring the effectiveness of said treatment through comparison of said measured erythrocyte potassium levels with said predetermined erythrocyte potassium level threshold.

41. The method of claim 40, wherein said individual is a human being over 20 years of age.

42. The method of claim 41, wherein said predetermined erythrocyte potassium level threshold is approximately 90 mmol/L per cell.

43. The method of claim 42, wherein said approximately 90 mmol/L per cell is 90 mmol/L per cell.

44. The method of claim 40, wherein said individual is a human being under 21 years of age.

45. The method of claim 44, wherein said predetermined erythrocyte potassium level threshold is approximately 94 mmol/L per cell.

46. The method of claim 45, wherein said approximately 94 mmol/L per cell is 94 mmol/L per cell.

47. The method of claim 40, wherein said treatment comprises a life-style modification.

48. The method of claim 40, wherein said treatment comprises a pharmacological treatment.

49. The method of claim 47, wherein said life-style modification comprises one or more life-style modifications selected from the group consisting of a dietary change, a reduction in alcohol intake, an increase in aerobic activity, a reduction or elimination of nicotine intake, an adequate intake of dietary calcium and magnesium, and a reduction of sodium intake.

50. The method of claim 48, wherein said pharmacological treatment comprises one or more pharmacological treatments selected from the group consisting of administration of one or more potassium sparing drugs, administration of one or more aldosterone blocking drugs, administration of one or more potassium oral supplements, and administration of one or more anti-hypertensive drugs.

51. A method of preventing the onset of hypertension, comprising
administering to an individual at risk for developing hypertension a treatment configured to increase said individual's erythrocyte potassium level above a predetermined erythrocyte potassium level threshold,
obtaining at least one measurement of said individual's erythrocyte potassium levels during the course of said treatment, and
monitoring the effectiveness of said treatment through comparison of said measured erythrocyte potassium levels with said predetermined erythrocyte potassium level threshold.

52. The method of claim 51, wherein said individual is a human being over 20 years of age.

53. The method of claim 52, wherein said predetermined erythrocyte potassium level threshold is approximately 90 mmol/L per cell.

54. The method of claim 53, wherein said approximately 90 mmol/L per cell is 90 mmol/L per cell.

55. The method of claim 51, wherein said individual is a human being under 21 years of age.

56. The method of claim 55, wherein said predetermined erythrocyte potassium level threshold is approximately 94 μmmol/L per cell.

57. The method of claim 56, wherein said approximately 94 mmol/L per cell is 94 mmol/L per cell.

58. The method of claim 51, wherein said treatment comprises a life-style modification.

59. The method of claim 51, wherein said treatment comprises a pharmacological treatment.

60. The method of claim 58, wherein said life-style modification comprises one or more life-style modifications selected from the group consisting of a dietary change, a reduction in alcohol intake, an increase in aerobic activity, a reduction or elimination of nicotine intake, an adequate intake of dietary calcium and magnesium, and a reduction of sodium intake.

61. The method of claim 59, wherein said pharmacological treatment comprises one or more pharmacological treatments selected from the group consisting of administration of one or more potassium sparing drugs, administration of one or more aldosterone blocking drugs, administration of one or more potassium oral supplements, and administration of one or more anti-hypertensive drugs.

62. A device for monitoring an individual's risk for hypertension, wherein said device is configured to:
a) receive a sample comprising erythrocytes from an individual;
b) measure the erythrocyte potassium level within said erythrocytes;
c) compare said measured erythrocyte potassium level with a predetermined erythrocyte potassium level threshold; and
d) report information pertaining to said comparison.

63. The device of claim 62, wherein said measured erythrocyte potassium levels above said predetermined erythrocyte potassium level threshold represent a low risk for hypertension, and wherein said measured erythrocyte potassium levels below said predetermined erythrocyte potassium level threshold represent a high risk for hypertension.

64. The device of claim 62, wherein said information is risk for hypertension.

65. The device of claim 62, wherein said information is an indication of whether said measured erythrocyte potassium level is above or below said predetermined erythrocyte potassium level threshold.

66. The device of claim 62, wherein said device is a hand-held device.

67. The device of claim 62, wherein said individual is a human being over 20 years of age.

68. The device of claim 67, wherein said predetermined erythrocyte potassium level threshold is approximately 90 mmol/L per cell.

69. The device of claim 68, wherein said approximately 90 mmol/L per cell is 90 mmol/L per cell.

70. The device of claim 62, wherein said individual is a human being under 21 years of age.

71. The device of claim 70, wherein said predetermined erythrocyte potassium level threshold is approximately 94 mmol/L per cell.

72. The device of claim 71, wherein said approximately 94 mmol/L per cell is 94 mmol/L per cell.

73. The device of claim 62, wherein said device is configured to utilize flame emission spectroscopy for purposes of measuring said individual's erythrocyte potassium level.

74. The device of claim 62, wherein said device is configured to utilize potassium selective electrodes for purposes of measuring said individual's erythrocyte potassium level.

75. The device of claim 62, the device has therein a processor that conducts said comparing.

76. The device of claim 75, wherein said processor is configured to interact with software configured to accomplish said comparing of said measured erythrocyte potassium level with said predetermined erythrocyte potassium level threshold.

77. The device of claim 62, wherein said reporting is wireless communication to the Internet.

78. The device of claim 62, wherein said reporting is wireless communication to a server.

79. The device of claim 62, wherein said reporting is a textually based message displayed on said device.

80. The device of claim 62, wherein said reporting is an audible message presented with said device.
